(19) United States
(12) Patent Application Publication
(10) Pub. No.: US 2026/0249104 A1
(43) Pub. Date: Aug. 27, 2026

(54) LASER POSITIONING DEVICE AND METHOD FOR BIPLANE RADIOTHERAPY

(71) Applicant: Sun Yat-sen Memorial Hospital, Sun Yat-sen University, Guangzhou (CN)

(72) Inventors: Zhuofei Bi, Guangzhou (CN); Jin Yang, Guangzhou (CN); Jiannan Fang, Guangzhou (CN); Yonglin Mao, Guangzhou (CN)

(21) Appl. No.: 19/190,865

(22) Filed: Apr. 28, 2025

(30) Foreign Application Priority Data
Feb. 26, 2025 (CN) 202510215581.9

(51) Int. Cl. A61N 5/10

(52) U.S. Cl. CPC A61N 5/1049; A61N 2005/105; A61N 2005/1061

(57) ABSTRACT

A laser positioning method for biplane radiotherapy is provided, which includes the following steps: a biplane laser positioning device of CT scanning equipment respectively forms double cross-shaped laser lines on the left side and the right side of a patient, and forms a pound sign-shaped laser line on the chest and abdomen of the patient; according to the results of CT scanning and positioning, mark lines are made on the patient's body surface; setup: the linear accelerator is provided with a biplane laser positioning device which is the same as the CT scanning equipment, and the setup is completed by overlapping the laser lines formed on the patient's body surface by the biplane laser positioning device of the linear accelerator with the marking lines made on the patient's body surface; and treatment is performed.

### CROSS-REFERENCE TO RELATED APPLICATIONS

This application claims priority to Chinese Patent Application No. 202510215581.9, filed on Feb. 26, 2025, the contents of which are hereby incorporated by reference.

### TECHNICAL FIELD

The disclosure relates to the field of medical instruments, and in particular to a laser positioning device for biplane radiotherapy and a method thereof.

### BACKGROUND

At present, the external laser positioning/setup system of radiotherapy equipment is generally composed of three groups of laser lamps, which are installed on the left and right walls and ceilings of CT, accelerator and other equipment respectively. The three groups of laser lines transmitted by the three groups of laser lamps construct three laser beam flow planes in space, namely, horizontal plane, transverse plane and sagittal plane, and establish a three-dimensional space radiotherapy coordinate system.

Taking the patient's supine position as a reference, the sagittal plane of the treatment coordinate system is constructed by the vertical laser lines transmitted by the laser lamp on the ceiling; the coronal plane is constructed by the horizontal laser lines transmitted by the left and right laser lamps, and the horizontal laser lines on the left and right sides coincide with each other; the transverse plane is constructed by vertical laser lines transmitted by the left and right laser lamps, and the vertical laser lines on the left and right sides overlap with each other; the sagittal plane, coronal plane and transverse plane are perpendicular to each other.

Due to the need of treatment positioning and setup, the vertical laser line in sagittal plane of radiotherapy CT simulation positioning equipment may move left and right; the horizontal laser lines on the left and right sides of the coronal plane may move in the abdomen and back direction; the vertical laser lines on the left and right sides of the transverse plane may move in the head and foot direction.

Three groups of laser lines converge on the patient and are displayed as a left cross-shaped laser line, a right cross-shaped laser line and a middle cross-shaped laser line. The left and right cross-shaped laser planes are completely overlapped to locate the position of the patient in the head and foot direction and the abdomen and back direction, and the top laser plane is perpendicular to the left and right laser planes to determine the position of the patient in the left and right direction. The left and right cross-shaped laser lines may mark the position of the patient in the head and foot direction and the abdomen and back direction, and then the direction of tumor entry and exit and the spatial position of the height of the treatment bed is capable of being controlled. The position of the patient in the left and right direction is capable of being determined by the top cross-shaped laser line, so as to determine the spatial position of the tumor in the left and right direction. Although the existing laser positioning method is simple and feasible, the positioning accuracy is not high because it is a single plane in the abdomen and back direction, the head and foot direction and the left and right direction, which may not effectively avoid the problem of trunk rotation and may not better determine the patient's body position.

### SUMMARY

The disclosure aims at overcoming the shortcomings of the prior art, and provides a laser positioning device for biplane radiotherapy and a method thereof, which are used for solving the problems that the existing laser positioning device and method have low positioning accuracy, may not effectively avoid trunk rotation and may not better determine the patient's body position.

The technical scheme adopted by the disclosure is as follows: a laser positioning method for biplane radiotherapy, including the following steps:
S1, CT scanning and positioning, where CT scanning equipment includes a laser positioning system, and the laser positioning system includes a left laser plane, a right laser plane and a top laser plane; the left laser plane and the right laser plane are respectively provided with a beam of parallel fan-shaped laser line and two beams of vertical fan-shaped laser lines, and double cross-shaped laser lines are formed on left and right sides of a patient; the top laser plane is provided with two fan-shaped laser lines perpendicular to vertical laser lines in the left laser plane and the right laser plane respectively, and together with the two beams of vertical laser lines in the left laser plane and the right laser plane, a pound sign-shaped laser line is formed in chest and abdomen of the patient;
S2, making marking lines on the patient's body surface according to results of the CT scanning and positioning;
S3, setup: a linear accelerator includes a same laser positioning system as a CT machine, and by adjusting the patient's position on the linear accelerator, the marking lines on the body surface are aligned with the laser positioning line of the linear accelerator; and
S4, performing treatment.

The laser positioning system on the CT scanning equipment forms double cross-shaped laser lines on both sides of the patient and a pound sign-shaped laser line on the chest and abdomen of the patient, and the doctor makes corresponding marking lines on the patient's body surface according to the CT scanning results. The linear accelerator is equipped with the same laser positioning system as the CT scanning equipment, so that the laser lines transmitted by the laser positioning system on the linear accelerator coincide with the marking lines on the patient's body surface respectively, and the patient's setup may be controlled during radiotherapy.

Both CT scanning equipment and linear accelerator adopt biplane laser positioning system, which may better control the setup error of patients during radiotherapy. By using biplane laser positioning method, there are nearly twice as many laser lines and marking lines on the patient's body surface, which may not only reduce the translation errors of the left and right direction, the abdomen and back direction and the head and foot direction, but also further control the errors in the head and foot rotation and left and right rotation of the patient, which will double the setup accuracy of the patient.

Further, in step S1, the parallel laser line and two beams of vertical laser lines on the left laser plane coincide with the parallel laser line and two beams of vertical laser lines on the right laser plane respectively.

The parallel laser line of the left laser plane coincides with the parallel laser line of the right laser plane to form a parallel laser line, which is capable of locating the height of the tumor in the patient's abdomen and back direction. If the parallel laser line of the left laser plane and the parallel laser line of the right laser plane do not coincide, two horizontal laser lines with different heights will be formed, which is not conducive to locating the height of the tumor in the abdomen and back direction of the patient. The two beams of vertical laser lines on the left laser plane coincide with the two beams of vertical laser lines on the right laser plane to form two vertical laser lines, which may be used to locate the position of the tumor in the the patient's head and foot direction. If the two beams of vertical laser lines on the left laser plane do not coincide with the two beams of vertical laser lines on the right laser plane, four vertical laser lines will appear in the patient's head and foot direction, and the positioning range in the patient's head and foot direction will be enlarged, which is not conducive to tumor positioning.

Further, the marking lines made in step S2 include two connected cross-shaped marking lines on a left side and a right side and a pound sign-shaped marking line on the chest and abdomen of the patient.

The laser positioning system of CT scanning equipment forms double cross-shaped laser lines on both sides of the patient and a pound sign-shaped laser line on the chest and abdomen. The laser positioning system on the linear accelerator is the same as the laser positioning system of CT scanning equipment, which will also form double cross-shaped laser lines on both sides of the patient and a pound sign-shaped laser line on the chest and abdomen. Double cross-shaped marking lines are made on the left and right sides of the patient, and a pound sign-shaped marking line is made on the chest and abdomen to make them consistent with the laser lines transmitted by the CT scanning equipment and the laser positioning system on the linear accelerator, so that the patient's setup may be controlled quickly and accurately.

Further, the setup in step S3 includes the following steps:
S31, adjusting vertical heights of the left laser plane and the right laser plane so that the laser lines parallel to a ground coincide with horizontal lines of double cross-shaped marking lines on the left and right sides of the patient;
S32, adjusting horizontal directions of the left laser plane and the right laser plane, so that two laser lines perpendicular to the ground respectively coincide with two vertical lines of the double cross-shaped marking lines on the left and right sides of the patient; and
S33, adjusting a horizontal direction of the top laser plane so that two beams of laser lines are respectively coincident with two vertical lines of the pound sign-shaped laser line.

The left laser plane and the right laser plane of the linear accelerator coincide with the double cross-shaped marking lines on the left and right sides of the patient respectively, so that the height of the left and right sides of the patient and the tilt angle in the head and foot direction are capable of being located. Taking the left side as an example, the horizontal laser line of the left laser plane of the linear accelerator coincides with the horizontal line of the double cross-shaped marking line on the left side of the patient, so that the vertical height of the left side of the patient is capable of being located. The two beams of vertical laser lines on the left laser plane of the linear accelerator coincide with the two vertical lines of the double cross-shaped marking line on the left side of the patient, so that the tilt angle of the patient in the head and foot direction is capable of being located. In the existing laser positioning system, the left laser plane and the marking line on the patient's left side are both in the shape of a single cross. When the left laser plane of the linear accelerator coincides with the cross-shaped marking line on the left side of the patient, there may be that the patient's head and foot direction on the linear accelerator is inclined relative to the head and foot direction on the CT machine, while the biplane laser positioning method may prevent this from happening. If the head and foot direction of the patient on the linear accelerator is inclined relative to the head and foot direction on the CT machine, only one of the two beams of vertical laser lines on the left laser plane coincides with the vertical line of the double cross-shaped line on the left side of the patient. The two beams of laser lines on the top laser plane coincide with the two vertical lines of the pound sign-shaped marking line respectively, which may control the rotation of the patient in the left and right direction, that is, the height of the left side of the patient relative to the right side of the patient.

Furthermore, in the laser positioning system, different colors are respectively used between two laser lines perpendicular to the ground in the left laser plane and the right laser plane, and between two laser lines in the top laser plane.

Different colors are used between two beams of laser lines perpendicular to the ground in the left laser plane and the right laser plane, and between two beams of laser lines on the top laser plane to distinguish and align easily and prevent confusion.

Further, the disclosure also provides a laser positioning device for biplane radiotherapy, including a left laser transmitter, a right laser transmitter and a top laser transmitter, where the left laser transmitter and the right laser transmitter respectively transmit a parallel fan-shaped laser line and two vertical fan-shaped laser lines; the top laser transmitter transmits two fan-shaped laser lines perpendicular to the vertical laser lines transmitted by the left laser transmitter and the right laser transmitter.

The biplane laser positioning device is arranged in the CT room and the radiotherapy room, and forms double cross-shaped and pound sign-shaped laser lines on both sides of the patient and the chest and abdomen respectively. Compared with the traditional single cross-shaped laser lines on both sides of the patient and the chest and abdomen, a group of reference lines are added in the head and foot direction and the left and right direction of the patient to form two planes, which may better inhibit the rotation of the trunk and improve the setup accuracy of radiotherapy.

Further, the left laser transmitter and the right laser transmitter respectively include a main laser transmitter and an auxiliary laser transmitter, where the main laser transmitter transmits a parallel laser line and a vertical laser line, the auxiliary laser transmitter transmits a vertical laser line, and a distance between the main laser transmitter and the auxiliary laser transmitter is capable of being adjusted.

Different patients' tumors have different sizes in the head and foot direction, so the distance between the main laser transmitter and the auxiliary laser transmitter may be adjusted to suit different patients. When the length of the tumor in the the patient's head and foot direction is long, adjusting the distance between the main laser transmitter and the auxiliary laser transmitter may improve the positioning accuracy.

Further, switches of the main laser transmitter and the auxiliary laser transmitter are capable of being independently controlled.

For patients whose tilt angle in the head and foot direction is easy to control, or whose tumor length in the head and foot direction is small, only the main laser transmitter may be used as needed to reduce the interference of the auxiliary laser transmitter.

Further, two beams of laser lines at the top are transmitted by two different laser transmitters, and a distance between the two laser transmitters is capable of being adjusted.

Different patients' tumors have different sizes in the left and right directions of patients, so the distance between the top two laser transmitters may be adjusted to suit different patients. When the length of the tumor in the patient's left and right direction is long, adjusting the distance between the two laser transmitters may improve the accuracy.

Furthermore, switches of two laser transmitters at the top are capable of being independently controlled.

When the height of the left and right sides of the patient is easy to control, or the length of the tumor in the left and right direction of the patient is small, only one of the laser transmitters may be turned on as needed to reduce the interference of the other laser transmitter.

Compared with the prior art, the laser positioning device for biplane radiotherapy has the following beneficial effects: the laser positioning device for biplane radiotherapy is applied to CT scanning equipment and linear accelerators, and double cross-shaped laser lines are formed on both sides of a patient, and a pound sign-shaped laser line is formed on the chest and abdomen of the patient. The biplane laser positioning system forms a three-dimensional space covering the gross tumor volume, which effectively solves the problem that a single plane may not restrain the rotation of the trunk. Using biplane laser positioning method, a group of reference lines are added in the head and foot direction and the left and right direction of the patient, which may not only reduce the translation errors in the left and right direction, the abdomen and back direction and the head and foot direction, but also control the errors in the head-foot rotation and the left-right rotation of the patient, which will double the setup accuracy of the patient. Different colors are used between the two beams of vertical laser lines in the left laser plane and the right laser plane, and between the two beams of laser lines in the top laser plane, so as to facilitate the distinction and alignment and prevent confusion. The distance between the main laser transmitter and the auxiliary laser transmitter, as well as between the two laser transmitters at the top, may be adjusted to make it suitable for different patients and improve the accuracy. The main laser transmitter, the auxiliary laser transmitter and the two laser transmitters at the top are respectively provided with independent control switches, which are capable of being adjusted according to the specific conditions of patients and are convenient to use.

### BRIEF DESCRIPTION OF THE DRAWINGS

FIG. 1 is a structural diagram of a laser positioning device for biplane radiotherapy according to the present disclosure.

FIG. 2 is a top view of the laser positioning device for biplane radiotherapy of the present disclosure.

FIG. 3 is a structural diagram showing that the existing laser positioning device of CT scanning equipment forms a cross-shaped laser line on the chest and abdomen of a patient.

FIG. 4 is a structural diagram showing that the existing laser positioning device of CT scanning equipment forms a cross-shaped laser line on the left side and chest and abdomen of the patient.

FIG. 5 is a structural diagram of a cross-shaped laser line formed on the chest and abdomen of a patient by the existing laser positioning device of a linear accelerator.

FIG. 6 is a structural diagram of the existing laser positioning device of the linear accelerator forming a cross-shaped laser line on the left side and chest and abdomen of the patient.

FIG. 7 is a step diagram of the laser positioning method for biplane radiotherapy according to the present disclosure.

FIG. 8 is a structural diagram of the laser positioning device for biplane radiotherapy of CT scanning equipment, which forms laser lines on the left side and chest and abdomen of the patient.

FIG. 9 is a step diagram of setup in the laser positioning method for biplane radiotherapy of the present disclosure.

FIG. 10 is a comparison diagram between the laser line formed on the left side of the patient by the laser positioning device of biplane radiotherapy of the linear accelerator and the marking line made on the left side of the patient.

FIG. 11 is a comparison diagram between the laser line formed on the chest and abdomen of the patient by the laser positioning device of biplane radiotherapy of the linear accelerator and the marking line made on the chest and abdomen of the patient.

### DESCRIPTION OF EMBODIMENTS

The drawings of the present disclosure are only for illustrative purposes and should not be construed as limiting the present disclosure. In order to better illustrate the following embodiments, some parts of the drawings will be omitted, enlarged or reduced, which do not represent the size of the actual product. It is understandable to those skilled in the art that some well-known structures in the drawings and their descriptions may be omitted.

### Embodiment 1

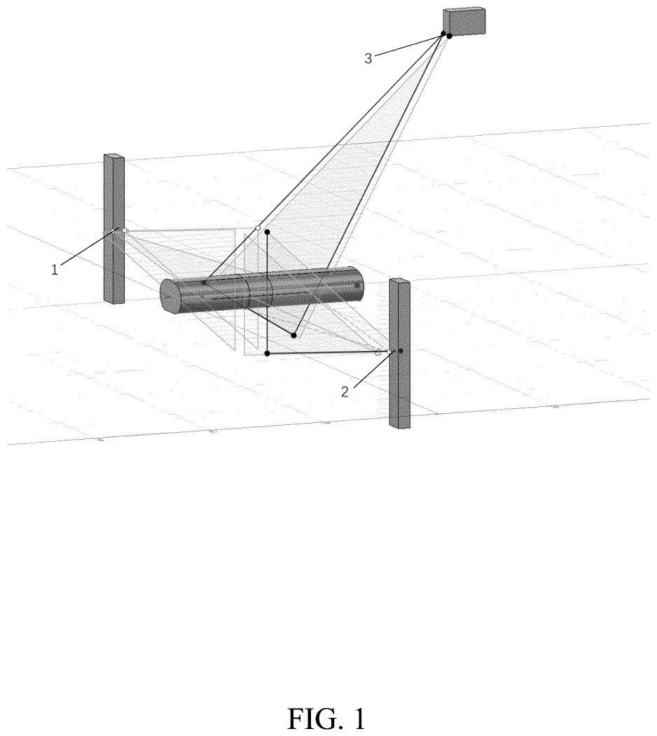

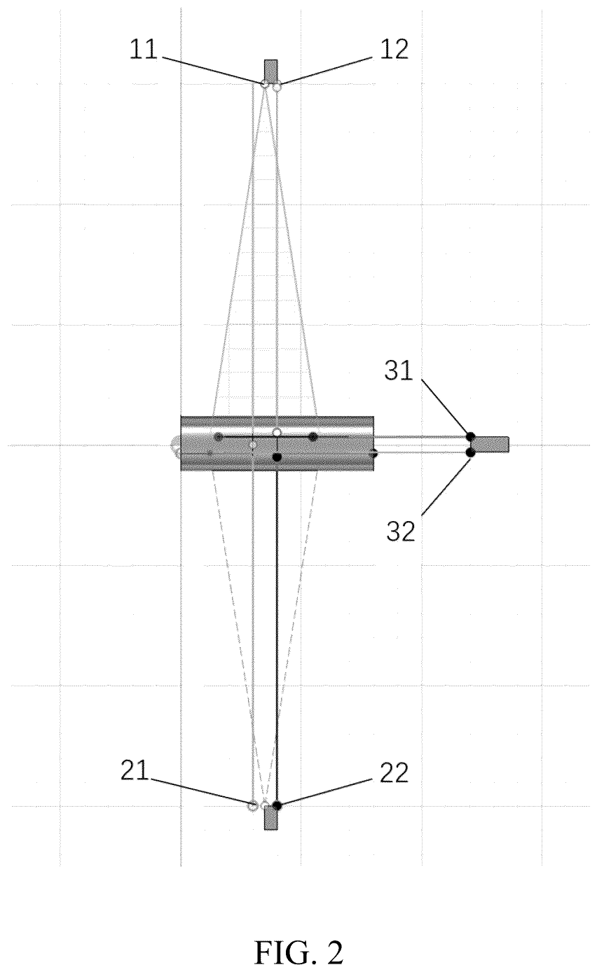

As shown in FIG. 1 and FIG. 2, this embodiment provides a laser positioning device for biplane radiotherapy, including a left laser transmitter 1, a right laser transmitter 2 and a top laser transmitter 3, which are respectively installed on the left and right side walls and ceilings of CT, linear accelerator and other equipment. With the patient's supine position as a reference, both the left laser transmitter 1 and the right laser transmitter 2 are capable of moving along the patient's head and foot direction and the abdomen and back direction, and the top laser transmitter 3 is capable of moving along the patient's left and right direction.

The left laser transmitter 1 and the right laser transmitter 2 respectively transmit a parallel fan-shaped laser line and two vertical fan-shaped laser lines; the top laser transmitter 3 transmits two fan-shaped laser lines perpendicular to the vertical laser lines transmitted by the left laser transmitter and the right laser transmitter.

Specifically, the left laser transmitter 1 includes a first main laser transmitter 11 and a first auxiliary laser transmitter 12; the right laser transmitter 2 includes a second main laser transmitter 21 and a second auxiliary laser transmitter 22. The first main laser transmitter 11 and the second main laser transmitter 21 respectively transmit a beam of parallel fan-shaped laser line and a beam of vertical fan-shaped laser line; the first auxiliary laser transmitter 12 and the second auxiliary laser transmitter 22 transmit a beam of vertical fan-shaped laser line; the fan-shaped laser lines transmitted by the first main laser transmitter 11 and the first auxiliary laser transmitter 12 form a double cross-shaped laser line on the left side of the patient; the fan-shaped laser lines transmitted by the second main laser transmitter 21 and the second auxiliary laser transmitter 22 form a double cross-shaped laser line on the right side of the patient. In addition, the vertical laser line transmitted by the first main laser transmitter 11 and the vertical laser line transmitted by the first auxiliary laser transmitter 12 adopt different colors respectively; the vertical laser line transmitted by the second main laser transmitter 21 and the vertical laser line transmitted by the second auxiliary laser transmitter 22 are different colors, respectively, so as to facilitate the distinction. The first auxiliary laser transmitter 12 and the second auxiliary laser transmitter 22 are respectively provided with guiding rails in the horizontal direction, so that the horizontal distance from the first main laser transmitter 11 and the second main laser transmitter 21 may be adjusted.

The top laser transmitter 3 includes a first laser transmitter 31 and a second laser transmitter 32; the first laser transmitter 31 and the second laser transmitter 32 respectively transmit a beam of fan-shaped laser line perpendicular to the vertical laser lines transmitted by the first main laser transmitter 11, the second main laser transmitter 21, the first auxiliary laser transmitter 12 and the second auxiliary laser transmitter 22; and the color of the laser line transmitted by the first laser transmitter 31 is different from that of the laser line transmitted by the second laser transmitter 32. A guiding rail is provided between the first laser transmitter 31 and the second laser transmitter 32, so that the distance between them is capable of being adjusted.

### Embodiment 2

The existing laser positioning methods are as follows: the laser positioning system of CT scanning equipment forms cross-shaped laser lines on both sides and chest and abdomen of the patient; according to the results of CT scanning and positioning, cross-shaped marking lines are made on the left side, right side and chest and abdomen of the patient. The cross-shaped laser lines formed by the laser positioning system of the linear accelerator on both sides and chest and abdomen of the patient coincide with the cross-shaped marking lines on the left side, right side and chest and abdomen of the patient. However, because the human body is flexible, any part may tilt or rotate. For example, when the cross-shaped marking lines and the above parts of the patient are not tilted or rotated, but the lower parts of the cross-shaped marking lines are tilted or rotated, the laser lines transmitted by the laser positioning system of the linear accelerator still coincide with the marking lines made on the patient's body surface, but in fact, the patient's position on the linear accelerator and on the CT scanning equipment has changed. If the tilted or rotated part is close to the tumor, it may drive the tumor in the patient to shift, thus affecting the therapeutic effect or accidentally injuring the normal tissue of the patient.

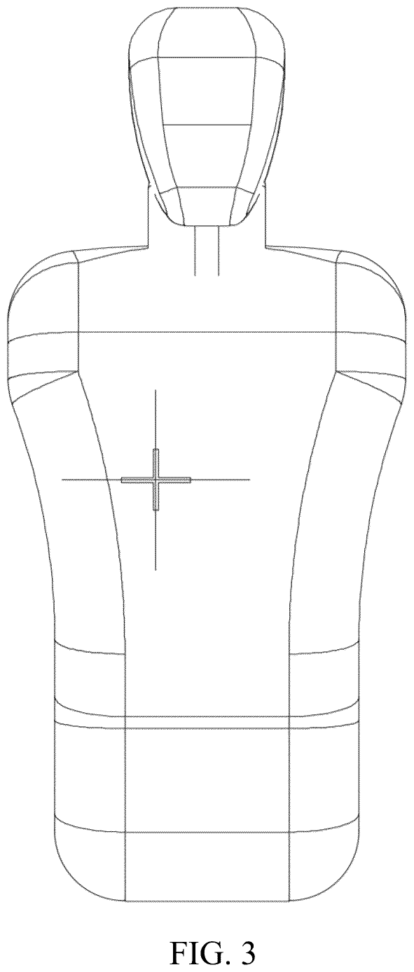

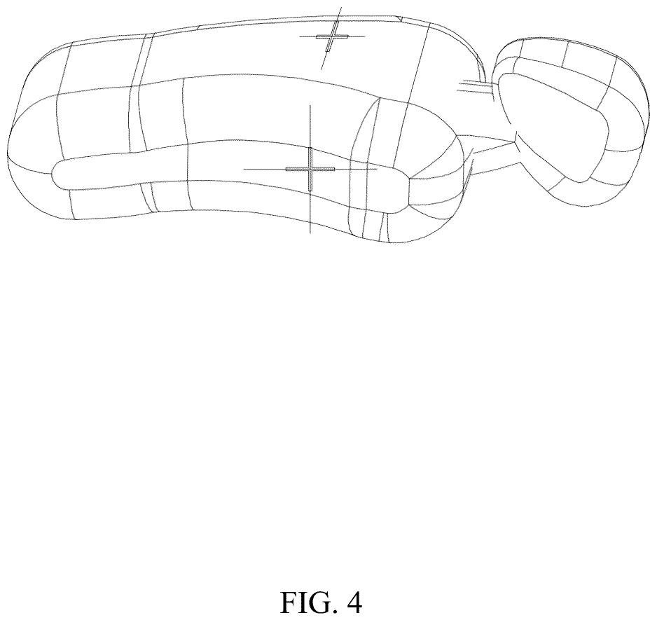

As shown in FIG. 3 and FIG. 4, the laser positioning system of CT scanning equipment forms a cross-shaped laser line on the left side, right side and chest and abdomen of the patient respectively. The marking line made on the left side of the patient is consistent with the cross-shaped laser lines formed by the laser positioning system on the left side, right side and chest and abdomen of the patient.

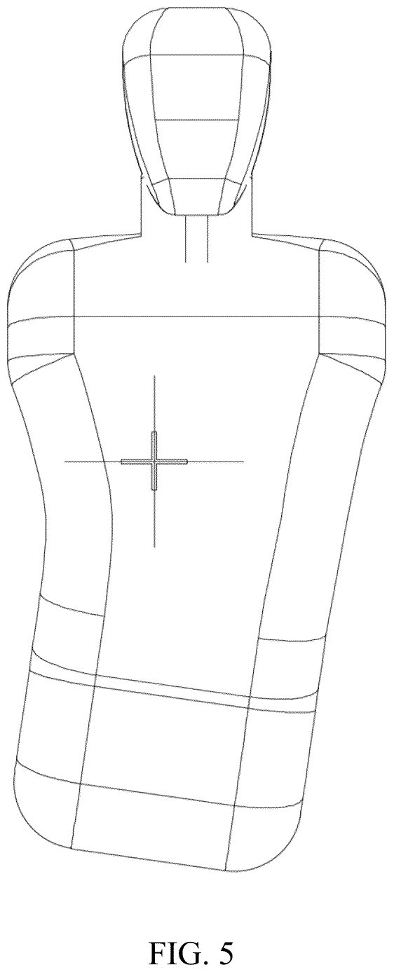

As shown in FIG. 5, the cross-shaped laser lines formed by the laser positioning system of the linear accelerator on the patient's left side, right side and chest and abdomen coincide with the cross-shaped marking lines made on the patient's left side, right side and chest and abdomen, but the patient's position at this time has changed compared with that on the CT scanning equipment, and the abdomen has tilted in the head and foot direction.

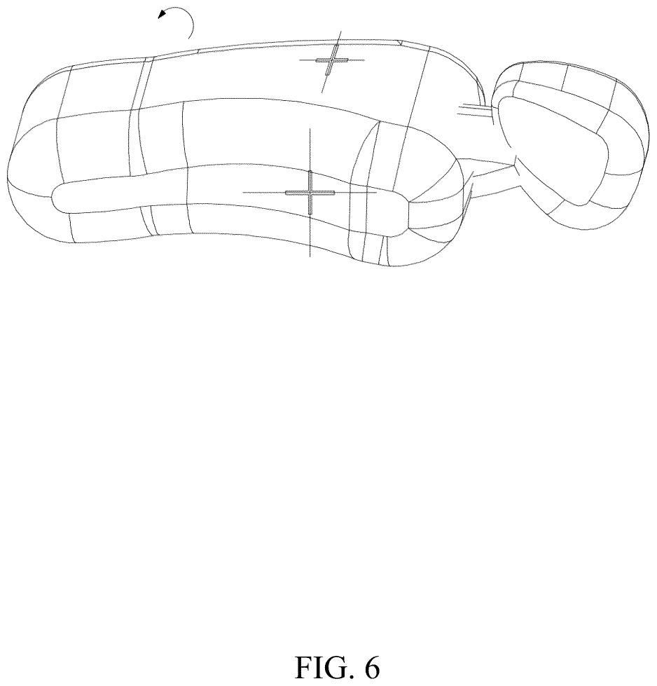

As shown in FIG. 6, the laser positioning system of the linear accelerator forms a cross-shaped laser line on the chest and abdomen of the patient, which coincides with the cross-shaped marking line of the chest and abdomen of the patient, but the position of the patient at this time has changed compared with that on the CT scanning equipment, and the abdomen has rotated in the left and right direction.

It can be seen that the existing laser positioning method may not control the patient's tilt in the head and foot direction and the rotation in the left and right directions. Although the laser lines formed on the patient's left side, right side and chest and abdomen coincide with the marking lines made on the patient's body surface, the patient's position on the linear accelerator has changed compared with that on the CT scanning equipment, and the positioning accuracy is low.

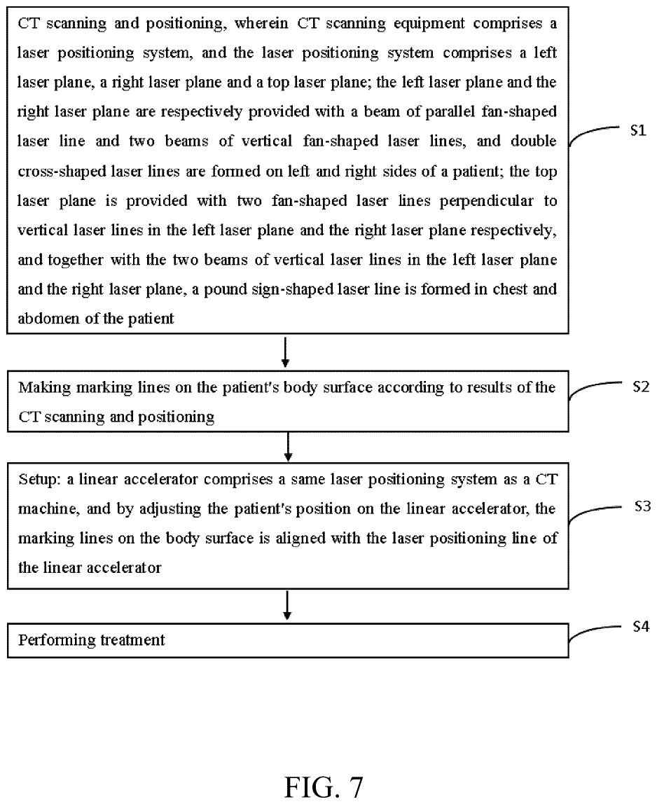

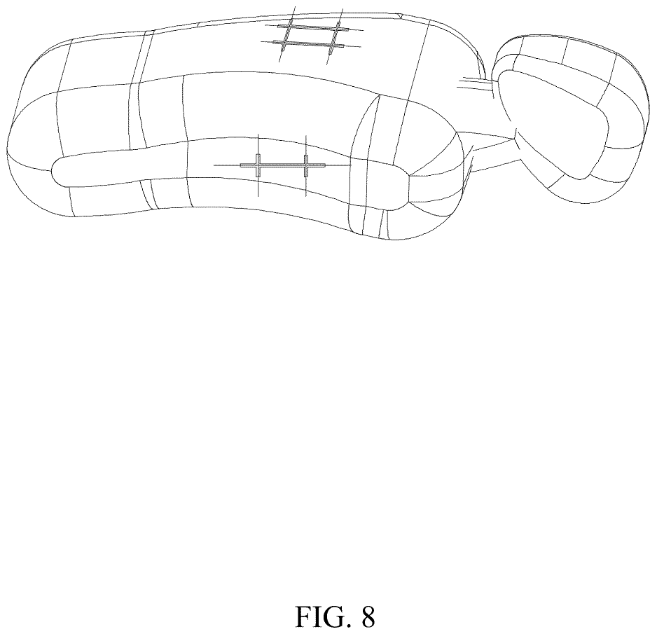

As shown in FIG. 7, this embodiment provides a laser positioning method for biplane radiotherapy, including the following steps:
S1, CT scanning and positioning, where CT scanning equipment includes a laser positioning system, and the laser positioning system includes a left laser plane, a right laser plane and a top laser plane. As shown in FIG. 8, the left laser plane and the right laser plane are respectively provided with a beam of parallel fan-shaped laser line and two beams of vertical fan-shaped laser lines, and double cross-shaped laser lines are formed on left and right sides of a patient; the top laser plane is provided with two fan-shaped laser lines perpendicular to vertical laser lines in the left laser plane and the right laser plane respectively, and together with the two beams of vertical laser lines in the left laser plane and the right laser plane, a pound sign-shaped laser line is formed in the chest and abdomen of the patient.

The parallel laser line and two beams of vertical laser lines on the left laser plane coincide with the parallel laser line and two beams of vertical laser lines on the right laser plane respectively.
S2, making marking lines on the patient's body surface according to results of the CT scanning and positioning.

As shown in FIG. 8, the marking lines are the same as the laser lines formed by the left laser plane, the right laser plane and the top laser plane on both sides of the patient and the chest and abdomen, that is, the left and right sides are double cross-shaped, and the chest and abdomen are pound sign-shaped.
S3, setup: a linear accelerator includes the same laser positioning system as a CT machine, and by adjusting the patient's position on the linear accelerator, the marking lines on the body surface are aligned with the laser positioning line of the linear accelerator.

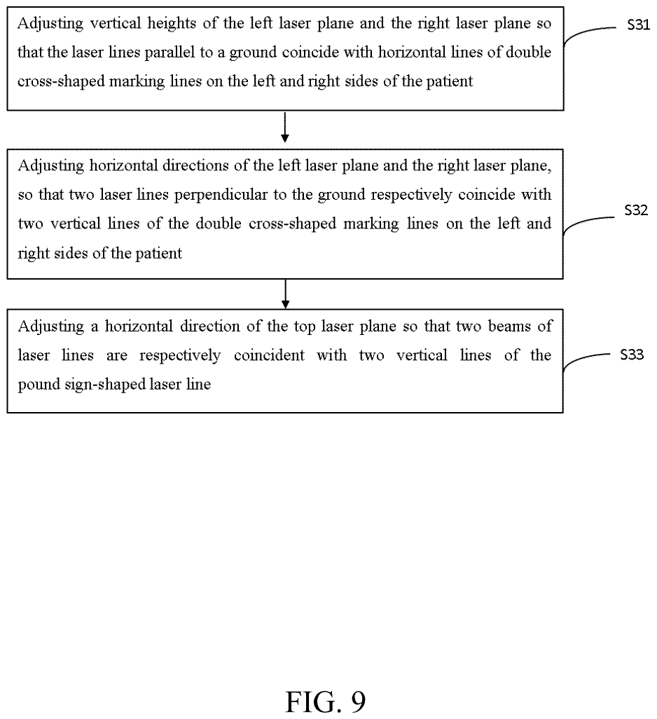

As shown in FIG. 9, the setup includes the following steps:
S31, adjusting vertical heights of the left laser plane and the right laser plane so that the laser lines parallel to the ground coincide with horizontal lines of double cross-shaped marking lines on the left and right sides of the patient;
S32, adjusting horizontal directions of the left laser plane and the right laser plane, so that two laser lines perpendicular to the ground respectively coincide with two vertical lines of the double cross-shaped marking lines on the left and right sides of the patient.

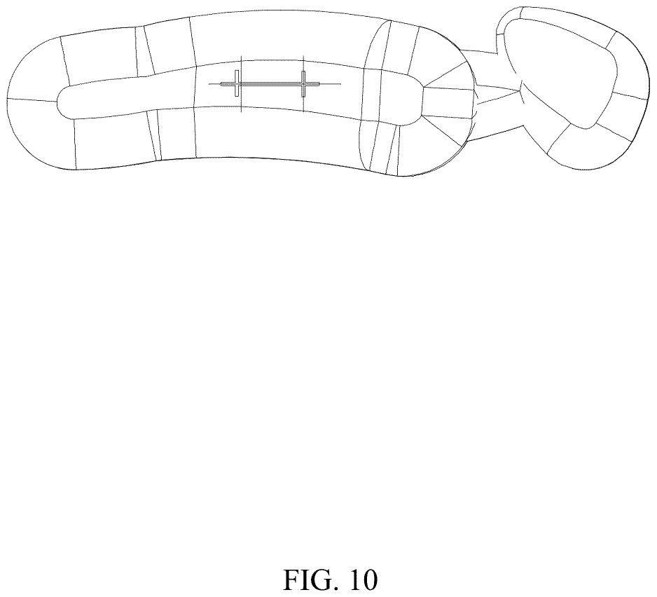

As shown in FIG. 10, the laser positioning system of the linear accelerator forms a double cross-shaped laser line on the left side of the patient. At this time, the vertical laser line near the patient's head coincides with the vertical line near the head in the double cross-shaped marking line, and the vertical laser line far from the patient's head does not coincide with the vertical line far from the head in the double cross-shaped marking line, and is within the two vertical lines of the double cross-shaped marking line, which shows that the patient's abdomen has tilted to the right in the head and foot direction. At this time, the patient's abdomen needs to be adjusted to the left, so that the vertical line far from the head in the double cross-shaped marking line on the left side of the patient coincides with the vertical laser line far from the head in the laser positioning system. On the contrary, if the vertical laser line far away from the patient's head is outside the two vertical lines of the double cross-shaped marking line, it means that the patient's abdomen has tilted to the left in the head and foot direction, and at this time, the patient's abdomen needs to be adjusted to the right. The adjustment method of the patient's right position is the same as that of the left side.
S33, adjusting a horizontal direction of the top laser plane so that two beams of laser lines are respectively coincident with two vertical lines of the pound sign-shaped laser line.

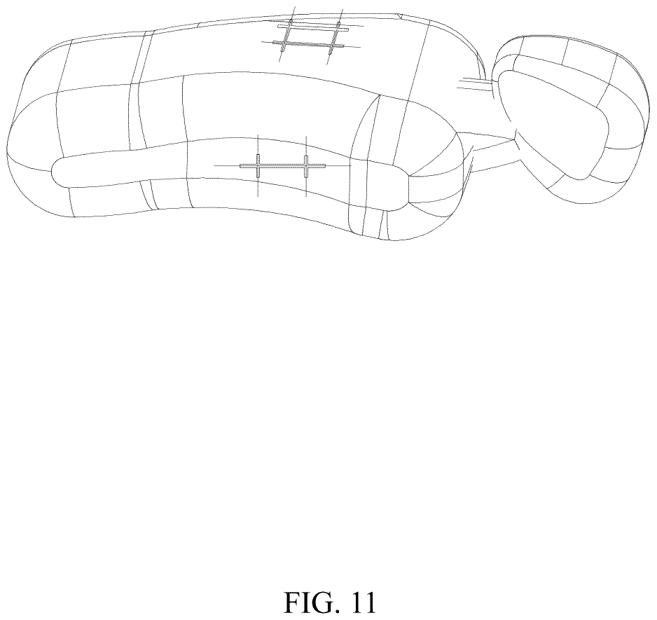

As shown in FIG. 11, the laser positioning system of the linear accelerator forms a pound sign-shaped laser line on the chest and abdomen of the patient. At this time, the laser line on the left side of the patient coincides with the vertical line on the left side of the pound sign-shaped marking line, and the laser line on the right side does not coincide with the vertical line on the right side, and is outside the two vertical lines of the pound sign-shaped marking line, which shows that the patient's right side rotates upwards. At this time, the patient's right side needs to be adjusted downward so that the vertical line on the patient's right side coincides with the laser line on the right side. On the contrary, if the laser line on the patient's right side is within two vertical lines of the pound sign-shaped marking line, it means that the patient's right side rotates downward, and at this time, the patient's right side needs to be adjusted upward.
S4, performing treatment.

Obviously, the above-mentioned embodiment of the present disclosure is only an example for clearly explaining the technical scheme of the present disclosure, and is not a limitation of the specific embodiment of the present disclosure. Any modification, equivalent substitution, improvement, etc. made within the spirit and principle of the claims of the present disclosure should be included in the protection scope of the claims of the present disclosure.

What is claimed is:

1. A laser positioning method for biplane radiotherapy, comprising following steps:
S1, CT scanning and positioning, wherein CT scanning equipment comprises a laser positioning system, and the laser positioning system comprises a left laser plane, a right laser plane and a top laser plane; the left laser plane and the right laser plane are respectively provided with a beam of parallel fan-shaped laser line and two beams of vertical fan-shaped laser lines, and double cross-shaped laser lines are formed on left and right sides of a patient;
the top laser plane is provided with two fan-shaped laser lines perpendicular to vertical laser lines in the left laser plane and the right laser plane respectively, and together with the two beams of vertical laser lines in the left laser plane and the right laser plane, a pound sign-shaped laser line is formed in chest and abdomen of the patient;
S2, making marking lines on the patient's body surface according to results of the CT scanning and positioning;
S3, setup: a linear accelerator comprises a same laser positioning system as a CT machine, and by adjusting the patient's position on the linear accelerator, the marking lines on the body surface are aligned with the laser positioning line of the linear accelerator; and
S4, performing treatment.

2. The laser positioning method for the biplane radiotherapy according to claim 1, wherein in step S1, the parallel laser line and two beams of vertical laser lines on the left laser plane coincide with the parallel laser line and two beams of vertical laser lines on the right laser plane respectively.

3. The laser positioning method for the biplane radiotherapy according to claim 1, wherein the marking lines made in step S2 comprise two connected cross-shaped marking lines on a left side and a right side and a pound sign-shaped marking line on the chest and abdomen of the patient.

4. The laser positioning method for the biplane radiotherapy according to claim 3, wherein the setup in step S3 comprises the following steps:
S31, adjusting vertical heights of the left laser plane and the right laser plane so that the laser lines parallel to a ground coincide with horizontal lines of double cross-shaped marking lines on the left and right sides of the patient;
S32, adjusting horizontal directions of the left laser plane and the right laser plane, so that two laser lines perpendicular to the ground respectively coincide with two vertical lines of the double cross-shaped marking lines on the left and right sides of the patient; and
S33, adjusting a horizontal direction of the top laser plane so that two beams of laser lines are respectively coincident with two vertical lines of the pound sign-shaped laser line.

5. The laser positioning method for the biplane radiotherapy according to claim 1, wherein in the laser positioning system, different colors are respectively used between two laser lines perpendicular to the ground in the left laser plane and the right laser plane, and between two laser lines in the top laser plane.

6. A laser positioning device for biplane radiotherapy, comprising a left laser transmitter, a right laser transmitter and a top laser transmitter, wherein the left laser transmitter and the right laser transmitter respectively transmit a parallel fan-shaped laser line and two vertical fan-shaped laser lines; the top laser transmitter transmits two fan-shaped laser lines perpendicular to the vertical laser lines transmitted by the left laser transmitter and the right laser transmitter.

7. The laser positioning device for the biplane radiotherapy according to claim 6, wherein the left laser transmitter and the right laser transmitter respectively comprise a main laser transmitter and an auxiliary laser transmitter, wherein the main laser transmitter transmits a parallel laser line and a vertical laser line, the auxiliary laser transmitter transmits a vertical laser line, and a distance between the main laser transmitter and the auxiliary laser transmitter is capable of being adjusted.

8. The laser positioning device for the biplane radiotherapy according to claim 7, wherein switches of the main laser transmitter and the auxiliary laser transmitter are capable of being independently controlled.

9. The laser positioning device for the biplane radiotherapy according to claim 6, wherein two beams of laser lines at the top are transmitted by two different laser transmitters, and a distance between the two laser transmitters is capable of being adjusted.

10. The laser positioning device for the biplane radiotherapy according to claim 9, wherein switches of two laser transmitters at the top are capable of being independently controlled.
